(19) United States
(12) Patent Application Publication
(10) Pub. No.: US 2024/0123258 A1
(43) Pub. Date: Apr. 18, 2024

(54) CLOUD-BASED DOSE VERIFICATION

(71) Applicant: Elekta, Inc., Atlanta, GA (US)

(72) Inventor: Qunxi WANG, Shanghai (CN)

(21) Appl. No.: 17/755,481

(22) PCT Filed: Nov. 7, 2020
(86) PCT No.: PCT/IB2020/000923

§ 371 Date: Apr. 29, 2022

(30) Foreign Application Priority Data
Nov. 8, 2019 (CN) 201911087723.9

(51) Int. Cl. A61N 5/10; G16H 20/40; G16H 30/20; H04L 9/40

(52) U.S. Cl. CPC A61N 5/1071; A61N 5/1039; A61N 5/1064; G16H 20/40; G16H 30/20; H04L 63/102

(57) ABSTRACT

Systems (500) and methods (700) for verifying a primary dose profile generated by a radiation machine using cloud-based services are disclosed. An exemplary system (500) can include a cloud (530,600) that provides cloud-based services, and a user interface (136) that enables multi-tenant access to the cloud-based services. A file service (610) can extract from a patient (501) DICOM file image information and information about a radiation machine. A dose engine service (620) can determine a secondary radiation dose profile by applying a dose algorithm (624) to the image and the radiation machine information. The applied dose algorithm (624) can be different from the dose algorithm used by the radiation machine to generate the primary dose profile. A dose evaluation service (630) can use the secondary radiation dose profile to verify accuracy of the primary dose profile based on a consistency indicator between the primary and secondary dose profiles.

### CLAIM FOR PRIORITY

This application claims the benefit of priority of Chinese Application Serial No. 201911087723.9, filed Nov. 8, 2019, which is hereby incorporated by reference in its entirety.

### TECHNICAL FIELD

This document relates generally to dose management in a radiation therapy treatment system, and more particularly, to systems and methods of verifying radiation dose profile using a cloud service.

### BACKGROUND

Radiation therapy (or “radiotherapy”) can be used to treat cancers or other ailments in mammalian (e.g., human and animal) tissue. One such radiotherapy technique is provided using a linear accelerator (also referred to as “linac”), whereby a tumor is irradiated by high-energy particles (e.g., electrons, protons, ions, high-energy photons, and the like). The placement and dose of the radiation beam can be accurately controlled to ensure the tumor receives the prescribed radiation, and the placement of the beam should be such as to minimize damage to the surrounding healthy tissue, often called the organ(s) at risk (OARs). A physician prescribes a predefined amount of radiation dose to the tumor and surrounding organs similar to a prescription for medicine. Generally, ionizing radiation in the form of a collimated beam is directed from an external radiation source toward a patient.

A specified or selectable beam energy can be used, such as for delivering a diagnostic energy level range or a therapeutic energy level range. Modulation of a radiation beam can be provided by one or more attenuators or collimators, such as a multi-leaf collimator (MLC). The intensity and shape of the radiation beam can be adjusted by collimation to avoid damaging healthy tissue (e.g., OARs) adjacent to the targeted tissue by conforming the projected beam to a profile of the targeted tissue.

Treatment planning is a process involving determination of specific radiotherapy parameters for implementing a treatment goal under the constraints. Examples of the radiotherapy parameters include radiation beam angles, dose intensity level, dose distribution, etc. The radiation dose can be calculated using a software model. The outcome of the treatment planning process is a radiotherapy treatment plan, hereinafter also referred to as a treatment plan or simply a plan. The treatment plan can be developed before radiotherapy is delivered, such as using one or more medical imaging techniques, such as images from X-rays, computed tomography (CT), nuclear magnetic resonance (MR), positron emission tomography (PET), single-photon emission computed tomography (SPECT), or ultrasound. A health care provider may use images of patient anatomy to identify a target tumor and the OARs near the tumor, delineate the target tumor that is to receive prescribed radiation dose, and similarly delineate nearby tissue such as organs at risk of damage from the radiation treatment. The delineation can be done manually, or by using an automated tool that assists in identifying or delineating the target tumor and OARs. A radiation therapy treatment plan can then be created using an optimization technique based on clinical and dosimetric objectives and constraints (e.g., the maximum, minimum, and fraction of dose of radiation to a fraction of the tumor volume, and like measures for the critical organs).

In some cases, the radiation dose calculated by a software model during the treatment planning process can be different from the actual dose measurement. The difference can be attributed to various factors including uncertainty of measurement, uncertainty of dose calculation, or uncertainty of dose delivery. To prevent or reduce the discrepancy between the calculated dose and the measurement dose, pre-treatment dose verification (also referred to as secondary dose check) has become an important quality assurance (QA) procedure and is routinely performed in a clinical facility. The dose verification process can include verifying the dose intensity map to radiation field coincidence by film. Proper dose verification can help prevent dose delivery errors to the patient, and ensure patient safety and therapy accuracy.

### OVERVIEW

MR-linac is a radiation treatment system that combines linac radiotherapy with diagnostic-level magnetic resonance imaging (MRI). The MR-linac can enable in-room MRI for anatomic and physiological treatment adaptation and response monitoring, and has a potential to reduce treatment margins with real-time visualization and target tracking. Tumors and surrounding tissue can be precisely located, their movement tracked, and treatment adapted in real time in response to changes in tumor position, shape, biology and spatial relationship to critical organs at the time of treatment.

The treatment planning procedure may include using a three-dimensional (3D) image of the patient to identify a target region (e.g., the tumor) and to identify critical organs near the tumor. Creation of a treatment plan can be a time-consuming process where a planner tries to comply with various treatment objectives or constraints (e.g., dose volume histogram (DVH), overlap volume histogram (OVH)), taking into account their individual importance (e.g., weighting) in order to produce a treatment plan that is clinically acceptable. The treatment plan is comprised of numerical parameters that specify the direction, cross-sectional shape, and intensity of each radiation beam. Once created, the treatment plan can be executed by positioning the patient in the treatment machine and delivering the prescribed radiation therapy directed by the optimized plan parameters. In some examples, The radiation therapy treatment plan can include dose “fractioning,” whereby a sequence of radiation treatments are provided over a predetermined period of time (e.g., 30-45 daily fractions), with each treatment including a specified fraction of a total prescribed dose. During treatment, the position of the patient and the position of the target tumor in relation to the treatment machine (e.g., linac) is very important in order to ensure the target tumor and not healthy tissue is irradiated.

The treatment planning system (TPS) can use a beam model (e.g., a software model) to determine dose metrics and other treatment parameters. A beam model can include parameters that describe, among other things, the energy distribution of radiation emitted from the radiation machine (e.g., a linac). The beam model parameter values can vary from one radiation machine to another, even radiation machines of the same model from the same manufacturer, at least because in each radiation machine there can be small differences, such as influence or energy provided by the radiation machine. Mechanical differences (e.g., mechanical dimensions or material properties) or differences in component values (e.g., electronic circuit component values) between the radiation machines can also contribute to the differences in beam model parameter values between different radiation machines.

To ensure patient safety and dose accuracy, radiation dose calculated by the TPS system can be verified before it is implemented in a treatment system and delivered to the patient. A radiation dose profile may include dose metrics that define the amount of radiation applied to target region (e.g., a tumor), the manner of delivering such radiation, and dose distribution in the target region and the OARs. Examples of the dose profile may include a percent depth dose (PDD) profile representing changes of relative dose with depth, a percentile radial dose (PRD) profile representing changes of relative dose with a radial distance, among others. Conventionally, a human expert (e.g., a radiation oncologist or a medical physicist) manually verifies that a calculated dose (e.g., a dose metric or a dose distribution) satisfies a pre-determined dosimetric verification criterion. The dosimetric verification criterion can be based on the analysis of a limited number of points in low-dose gradient areas, or the measurement of distances between isodose lines in high-dose gradient areas. The human expert can compare the desired dose and film measurement results by placing these transparency films side by side to visualize their discrepancy, or by superimposing these films of isodose curves onto planning results to check any differences or to verify consistency.

Manual dose verification, such as visual inspection and comparison of films, can sometimes be inconsistent and lead to interpretation errors. Additionally, although manual verification may be adequate for dose plans for two-dimensional (2D) conventional radiotherapy or some simple three-dimensional (3D) conformal radiotherapies, evolvement of more advanced radiation therapy techniques (e.g., intensity-modulated radiation therapy (IMRT) or volumetric-modulated arc therapy (VMAT)) generally adds more complexity to dose calculation and requires more complicated dose metrics and distribution representations. For example, the integrity of complexity of the IMRT dose delivery technique relies on quantification of the coincidence of the planned and delivered IMRT dose distributions. As such, manual verification of the doses generated by the TPS of advanced radiotherapy systems can be time-consuming and burdensome.

As an alternative or supplement to the manual dose verification, an automatic dose monitor can automatically compute a dose profile and compare it to the dose profile generated by the TPS of a radiation machine for dose verification. However, current automatic dose verification systems generally cannot verify dose profiles produced by different TPS systems of the radiotherapy equipment with different models or from different manufactures. Moreover, these automatic dose verification systems are generally incapable of, or not optimized for, simultaneously handling a high volume of dose verification requests such as from different hospitals or clinical facilities. The present inventor has recognized an unmet need for systems, devices, and methods of dose verification that can accommodate a large volume of dose profiles produced by a wide variety of TPS systems. Such a versatile dose verification system can be an integral part of an improved quality assurance (QA) system suited for the advanced radiotherapy such as IMRT or VMAT.

The present document discusses cloud-based systems, devices, and methods for verifying a primary dose profile generated by a radiation machine (e.g., a linac) for providing a radiotherapy to a subject. An exemplary system includes a cloud that can provide a suite of cloud-based services, and a user interface enables multi-tenant access to one or more of the cloud-based services. The cloud services include a file service that can receive patient image information and information about a radiation machine that generates the patient image. The patient image information and the radiation machine information can be extracted from a DICOM file. A dose engine service can determine a secondary radiation dose profile by applying a dose algorithm to the image and the radiation machine information. The applied dose algorithm can be different from the dose algorithm used by the radiation machine to generate the primary dose profile. A dose evaluation service can use the secondary radiation dose profile to verify accuracy of the primary dose profile based on a consistency indicator between the primary and secondary dose profiles.
Example 1 is a system for verifying a primary radiation dose profile generated by a radiation machine for providing a radiotherapy to a subject. The system comprises: a cloud-computing device or networked devices configured to provide cloud-based services including: a file service to receive (1) image information of the subject produced by the radiation machine and (2) information about the radiation machine; a dose engine service to determine a secondary radiation dose profile using the received image information and the radiation machine information; and a dose evaluation service to verify the primary radiation dose profile using the secondary radiation dose profile; and a user interface configured to enable a client access to one or more of the cloud-based services via a communication network, and to output the verification of the primary radiation dose profile to a user or a process.
In Example 2, the subject matter of Example 1 optionally includes the file service that can be configured to receive a DICOM file of the subject, and to parse the received DICOM file to extract the image information and the radiation machine information from the received DICOM file.
In Example 3, the subject matter of Example 2 optionally includes the file service that can be configured to extract treatment plan information from the parsed DICOM file, the treatment plan information including the primary radiation dose profile.
In Example 4, the subject matter of any one or more of Examples 2-3 optionally includes the DICOM file that can be a compressed or encrypted DICOM file. The file service can be configured to decompress or decrypt the DICOM file, and to parse the decompressed or decrypted DICOM file.
In Example 5, the subject matter of any one or more of Examples 2-4 optionally includes a cloud storage that can be configured to store the DICOM file of the subject, or the image information and the radiation machine information extracted from the DICOM file.
In Example 6, the subject matter of any one or more of Examples 1-5 optionally includes the radiation machine information received by the file service that can include one or more radiation beam parameters or one or more gantry parameters.
In Example 7, the subject matter of any one or more of Examples 1-6 optionally includes the dose engine service that can be configured to apply a secondary dose algorithm to one or more of the image information or the radiation machine information to determine the secondary radiation dose, the secondary dose algorithm different from a primary dose algorithm used by the radiation machine to calculate the primary radiation dose profile.
In Example 8, the subject matter of Example 7 optionally includes the user interface that can be configured to receive a user input of the secondary dose algorithm, or a user selection of the secondary dose algorithm from a plurality of candidate dose algorithms.
In Example 9, the subject matter of any one or more of Examples 1-8 optionally includes the dose evaluation service that can be configured to determine a consistency metric between the primary radiation dose profile and the secondary radiation dose profile, and to verify the primary radiation dose profile based on the determined consistency metric.
In Example 10, the subject matter of Example 9 optionally includes the dose consistency metric that can include a relative difference between the primary radiation dose profile and the secondary radiation dose profile, and the dose evaluation service can be configured to verify the primary radiation dose profile in response to the dose consistency metric falling within a specified range.
In Example 11, the subject matter of any one or more of Examples 1-10 optionally includes the cloud-computing device or networked devices that can include access points configured to support simultaneous access to the cloud-based services by two or more clients.
In Example 12, the subject matter of Example 11 optionally includes the cloud-based services that can further include a tenant management service configured to queue service requests from the two or more clients.
In Example 13, the subject matter of any one or more of Examples 1-12 optionally includes the cloud-computing device or networked devices that can be configured to generate a radiation treatment plan for the subject using a beam model based on the verification of the primary dose profile.
Example 14 is a method for verifying a primary radiation dose profile generated by a radiation machine for providing a radiotherapy to a subject. The method comprises, via a cloud-computing device or networked devices, steps of: receiving a DICOM file of the subject; parsing the received DICOM file and extracting image information and information about the radiation machine from the received DICOM file; determining a secondary radiation dose profile using the image information and the machine information; and verifying the primary radiation dose profile using the secondary radiation dose profile.
In Example 15, the subject matter of Example 14 optionally includes determining the secondary radiation dose profile that can include applying a secondary dose algorithm to one or more of the image information or the radiation machine information, the secondary dose algorithm different from a primary dose algorithm used by the radiation machine to calculate the primary radiation dose profile.
In Example 16, the subject matter of Example 15 optionally includes receiving a user input of the secondary dose algorithm, or a user selection of the secondary dose algorithm from a plurality of candidate dose algorithms.
In Example 17, the subject matter of any one or more of Examples 14-16 optionally includes verifying the primary radiation dose profile that can include determining a consistency metric between the primary radiation dose profile and the secondary radiation dose profile, and verifying the primary radiation dose profile in response to the determined consistency metric satisfying a condition.
In Example 18, the subject matter of Example 17 optionally includes queuing service requests from the two or more clients simultaneously accessing to the cloud-based services.
In Example 19, the subject matter of any one or more of Examples 14-18 optionally includes generating a radiation treatment plan for the subject using a beam model based on the verification of the primary dose profile.
Example 20 is a non-transitory machine-readable storage medium that includes instructions that, when executed by one or more processors of a machine, cause the machine to perform operations comprising: receiving a DICOM file of a subject, the DICOM file generated by a radiation machine for providing a radiotherapy to the subject; parsing the received DICOM file and extracting image information and information about the radiation machine from the received DICOM file; determining a secondary radiation dose profile using the image information and the machine information; and verifying the primary radiation dose profile using the secondary radiation dose profile.
In Example 21, the subject matter of Example 20 optionally includes the operation of determining the secondary radiation dose profile that can include applying a secondary dose algorithm to one or more of the image information or the radiation machine information, the secondary dose algorithm different from a primary dose algorithm used by the radiation machine to calculate the primary radiation dose profile.
In Example 22, the subject matter of Example 21 optionally includes operations that can include receiving a user input of the secondary dose algorithm, or a user selection of the secondary dose algorithm from a plurality of candidate dose algorithms.
In Example 23, the subject matter of any one or more of Examples 20-22 optionally includes the operation of verifying the primary radiation dose profile that can include determining a consistency metric between the primary radiation dose profile and the secondary radiation dose profile, and verifying the primary radiation dose profile in response to the determined consistency metric satisfying a condition.
In Example 24, the subject matter of Example 23 optionally includes the operations comprising queuing service requests from the two or more clients simultaneously accessing to the cloud-based services.
In Example 25, the subject matter of any one or more of Examples 23-24 optionally includes the operations comprising generating a radiation treatment plan using a beam model based on the verification of the primary dose profile.

The cloud-based dose verification system, device, and methods discussed herein may improve automated quality assurance (QA) in advanced radiation therapy such as IMRT or VMAT. Compared to the conventional manual or automatic dose verification approaches, the cloud-based dose verification as discussed herein has several advantages. First, it is versatile to verify dose profiles produced by a multitude of TPS systems of different models or made by different manufacturers. Second, it supports multi-tenant access to the cloud-bases services, allowing multiple tenants, such as from different hospitals or clinical facilities, to subscribe, and simultaneously access to various resources and cloud services, thereby reducing the cost of equipment and maintenance at each client end. Third, the cloud-based automatic dose verification can significantly reduce the workload of human dose verifiers, improve the accuracy and efficiency of dose verification, and increase data security and flexibility of data management.

The above is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example but not by way of limitation, various embodiments discussed in the present document.

FIG. 1 illustrates an exemplary radiotherapy system.

FIG. 2A illustrates an exemplary radiotherapy system that can provide a therapy beam.

FIG. 2B illustrates an exemplary combined system including a computed tomography (CT) imaging system and a radiation therapy system.

FIG. 3 illustrates a partially cut-away view of an exemplary combined system including a nuclear magnetic resonance (MR) imaging system and a radiation therapy system.

FIG. 4 illustrates an example of a dose profile generated by a therapy planning system.

FIG. 5 is a diagram illustrating an exemplary architecture of a cloud-based dose verification system.

FIG. 6 is a block diagram illustrating an exemplary cloud server configured to provide data storage and a suite of cloud-based services including dose verification.

FIG. 7 is a flow-chart illustrating an exemplary method of verifying a dose profile generated by a TPS system of a radiotherapy system using cloud services.

FIG. 8 illustrates generally a block diagram of an example machine upon which any one or more of the techniques (e.g., methodologies) discussed herein may perform.

### DETAILED DESCRIPTION

In the following detailed description, reference is made to the accompanying drawings which form a part hereof, and which is shown by way of illustration-specific embodiments in which the present disclosure may be practiced. These embodiments, which are also referred to herein as “examples,” are described in sufficient detail to enable those skilled in the art to practice the disclosure, and it is to be understood that the embodiments may be combined, or that other embodiments may be utilized and that structural, logical and electrical changes may be made without departing from the scope of the present disclosure. The following detailed description is, therefore, not be taken in a limiting sense, and the scope of the present disclosure is defined by the appended aspects and their equivalents.

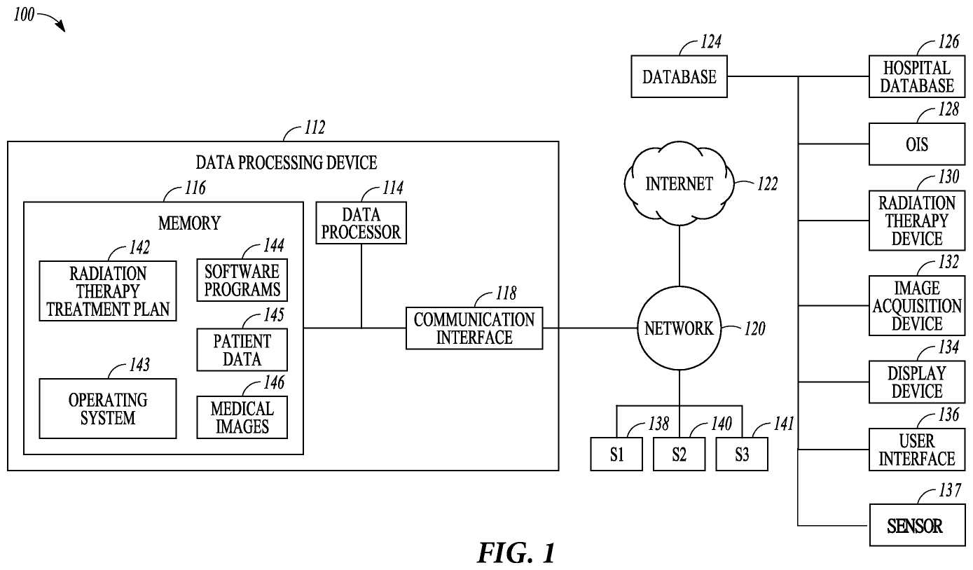

FIG. 1 illustrates an exemplary radiotherapy system 100 for providing radiation therapy to a patient. The radiotherapy system 100 includes a data processing device 112. The data processing device 112 may be connected to a network 120. The network 120 may be connected to the Internet 122. The network 120 can connect the data processing device 112 with one or more of a database 124, a hospital database 126, an oncology information system (OIS) 128, a radiation therapy device 130, an image acquisition device 132, a display device 134, and a user interface 136. The data processing device 112 can be configured to generate radiation therapy treatment plans 142 to be used by the radiation therapy device 130.

The data processing device 112 may include a memory device 116, a processor 114, and a communication interface 118. The memory device 116 may store computer-executable instructions, such as an operating system 143, a radiation therapy treatment plan 142 (e.g., original treatment plans, adapted treatment plans and the like), software programs 144, and any other computer-executable instructions to be executed by the processor 114. The memory device 116 may additionally store data, including medical images 146, patient data 145, and other data required to implement a radiation therapy treatment plan 142.

The software programs 144 can include one or more software packages that, when executed by a machine such as the data processor 114, can perform specific image processing and generating a radiation therapy treatment plan 142. In an example, the software programs 144 can convert medical images of one format (e.g., MRI) to another format (e.g., CT) by producing synthetic images, such as pseudo-CT images. For instance, the software programs 144 may include image processing programs to train a predictive model for converting a medical image from the medical images 146 in one modality (e.g., an MR image) into a synthetic image of a different modality (e.g., a pseudo CT image); alternatively, the trained predictive model may convert a CT image into an MR image. In another example, the software programs 144 may register the patient image (e.g., a CT image or an MR image) with that patient's dose distribution (also represented as an image) so that corresponding image voxels and dose voxels are associated appropriately by the network. In yet another example, the software programs 144 may substitute functions of the patient images such as signed distance functions or processed versions of the images that emphasize some aspect of the image information. Such functions might emphasize edges or differences in voxel textures, or any other structural aspect useful to neural network learning. The software programs 144 may substitute functions of the dose distribution that emphasize some aspect of the dose information. Such functions might emphasize steep gradients around the target or any other structural aspect useful to neural network learning.

In an example, the software programs 144 may generate projection images for a set of two-dimensional (2D) and/or 3D CT or MR images depicting an anatomy (e.g., one or more targets and one or more OARs) representing different views of the anatomy from a first gantry angle of the radiotherapy equipment. For example, the software programs 144 may process the set of CT or MR images and create a stack of projection images depicting different views of the anatomy depicted in the CT or MR images from various perspectives of the gantry of the radiotherapy equipment. In particular, one projection image may represent a view of the anatomy from 0 degrees of the gantry, a second projection image may represent a view of the anatomy from 45 degrees of the gantry, and a third projection image may represent a view of the anatomy from 90 degrees of the gantry. The degrees may be a position of the MLC relative to a particular axis of the anatomy depicted in the CT or MR images. The axis may remain the same for each of the different degrees that are measured.

In an example, the software programs 144 may generate graphical aperture image representations of MLC leaf positions at various gantry angles. These graphical aperture images are also referred to as aperture images. In particular, the software programs 144 may receive a set of control points that are used to control a radiotherapy device to produce a radiotherapy beam. The control points may represent the beam intensity, gantry angle relative to the patient position, and the leaf positions of the MLC, among other machine parameters. Based on these control points, a graphical image may be generated to graphically represent the beam shape and intensity that is output by the MLC at each particular gantry angle. The software programs 144 may align each graphical image of the aperture at a particular gantry angle with the corresponding projection image at that angle that was generated. The images are aligned and scaled with the projections such that each projection image pixel is aligned with the corresponding aperture image pixel.

In an example, the software programs 144 can include a treatment planning software for generating or estimating a graphical aperture image representation of MLC leaf positions at a given gantry angle for a projection image of the anatomy representing the view of the anatomy from the given gantry angle. The software programs 144 may further include a beam model that can simulate the radiation exiting the radiation machine and impinging upon the patient. The software programs 144 can compute machine parameters or control points for a given type of machine to output a beam from the MLC that achieves the same or similar estimated graphical aperture image representation of the MLC leaf positions. Namely, the treatment planning software may output an image representing an estimated image of the beam shape and intensity for a given gantry angle and for a given projection image of the gantry at that angle, and the function may compute the control points for a given radiotherapy device to achieve that beam shape and intensity.

In addition to the memory 116 storing the software programs 144, the software programs 144 may additionally or alternatively be stored on a removable computer medium, such as a hard drive, a computer disk, a CD-ROM, a DVD, a HD, a Blu-Ray DVD, USB flash drive, a SD card, a memory stick, or any other suitable medium; and the software programs 144 when downloaded to data processing device 112 may be executed by data processor 114.

The data processor 114 may be communicatively coupled to the memory 116, and the processor 114 may be configured to execute computer executable instructions stored therein. The processor 114 may send or receive medical images 146 to or from the memory 116. For example, the processor 114 may receive medical images 146 from the image acquisition device 132 via the communication interface 118 and network 120 to be stored in memory 116. The processor 114 may also send medical images 146 stored in memory 116 via the communication interface 118 to the network 120 be stored in the database 124 or the hospital database 126.

The data processor 114 may utilize the software programs 144 (e.g., a treatment planning software), along with the medical images 146 and patient data 145, to create the radiation therapy treatment plan 142. Medical images 146 may include information such as imaging data associated with a patient anatomical region, organ, or volume of interest segmentation data. Patient data 145 may include information such as (1) functional organ modeling data (e.g., serial versus parallel organs, appropriate dose response models, etc.); (2) radiation dosage data (e.g., DVH information); or (3) other clinical information about the patient and treatment (e.g., other surgeries, chemotherapy, previous radiotherapy, etc.).

In some examples, the processor 114 may utilize software programs 144 to generate intermediate data such as updated parameters to be used, for example, by a machine learning model, such as a neural network model; or generate intermediate 2D or 3D images, which may then subsequently be stored in memory 116. The processor 114 may subsequently then transmit the executable radiation therapy treatment plan 142 via the communication interface 118 to the network 120 to the radiation therapy device 130, where the radiation therapy plan will be used to treat a patient with radiation. In addition, the processor 114 may execute software programs 144 to implement functions such as image conversion, image segmentation, deep learning, neural networks, and artificial intelligence. For instance, the processor 114 may execute software programs 144 that train or contour a medical image; such software programs 144 when executed may train a boundary detector or utilize a shape dictionary.

The processor 114 may be a processing device, include one or more general-purpose processing devices such as a microprocessor, a central processing unit (CPU), a graphics processing unit (GPU), an accelerated processing unit (APU), or the like. More particularly, the processor 114 may be a complex instruction set computing (CISC) microprocessor, a reduced instruction set computing (RISC) microprocessor, a very long instruction Word (VLIW) microprocessor, a processor implementing other instruction sets, or processors implementing a combination of instruction sets. The processor 114 may also be implemented by one or more special-purpose processing devices such as an application specific integrated circuit (ASIC), a field programmable gate array (FPGA), a digital signal processor (DSP), a System on a Chip (SoC), or the like. As would be appreciated by those skilled in the art, in some embodiments, the processor 114 may be a special-purpose processor, rather than a general-purpose processor. The processor 114 may include one or more known processing devices, such as a microprocessor from the Pentium™, Core™, Xeon™, or Itanium® family manufactured by Intel™, the Turion™, Athlon™, Sempron™, Opteron™ FX™, Phenom™ family manufactured by AMD™, or any of various processors manufactured by Sun Microsystems. The processor 114 may also include graphical processing units such as a GPU from the GeForce®, Quadro®, Tesla® family manufactured by Nvidia™, GMA, Iris™ family manufactured by Intel™, or the Radeon™ family manufactured by AMD™. The processor 114 may also include accelerated processing units such as the Xeon Phi™ family manufactured by Intel™. The disclosed embodiments are not limited to any type of processor(s) otherwise configured to meet the computing demands of identifying, analyzing, maintaining, generating, and/or providing large amounts of data or manipulating such data to perform the methods disclosed herein. In addition, the term “processor” may include more than one processor (for example, a multi-core design or a plurality of processors each having a multi-core design). The processor 114 can execute sequences of computer program instructions, stored in memory 116, to perform various operations, processes, methods that will be explained in greater detail below.

The memory device 116 can store medical images 146. In some embodiments, the medical images 146 may include one or more MRI images (e.g., 2D MRI, 3D MRI, 2D streaming MRI, four-dimensional (4D) MRI, 4D volumetric MRI, 4D cine MRI, etc.), functional MRI images (e.g., fMRI DCE-MRI, diffusion MRI), CT images (e.g., 2D CT, cone beam CT, 3D CT, 4D CT), ultrasound images (e.g., 2D ultrasound, 3D ultrasound, 4D ultrasound), one or more projection images representing views of an anatomy depicted in the MRI, synthetic CT (pseudo-CT), and/or CT images at different angles of a gantry relative to a patient axis, PET images, X-ray images, fluoroscopic images, radiotherapy portal images, SPECT images, computer generated synthetic images (e.g., pseudo-CT images), aperture images, graphical aperture image representations of MLC leaf positions at different gantry angles, and the like. Further, the medical images 146 may also include medical image data, for instance, training images, and ground truth images, contoured images, and dose images. In an embodiment, the medical images 146 may be received from the image acquisition device 132. Accordingly, image acquisition device 132 may include an MRI imaging device, a CT imaging device, a PET imaging device, an ultrasound imaging device, a fluoroscopic device, a SPECT imaging device, an integrated linac and MRI imaging device, or other medical imaging devices for obtaining the medical images of the patient. The medical images 146 may be received and stored in any type of data or any type of format that the data processing device 112 may use to perform operations consistent with the disclosed embodiments.

The memory device 116 may be a non-transitory computer-readable medium, such as a read-only memory (ROM), a phase-change random access memory (PRAM), a static random access memory (SRAM), a flash memory, a random access memory (RAM), a dynamic random access memory (DRAM) such as synchronous DRAM (SDRAM), an electrically erasable programmable read-only memory (EEPROM), a static memory (e.g., flash memory, flash disk, static random access memory) as well as other types of random access memories, a cache, a register, a CD-ROM, a DVD or other optical storage, a cassette tape, other magnetic storage device, or any other non-transitory medium that may be used to store information including image, data, or computer executable instructions (e.g., stored in any format) capable of being accessed by the processor 114, or any other type of computer device. The computer program instructions can be accessed by the processor 114, read from the ROM, or any other suitable memory location, and loaded into the RAM for execution by the processor 114. For example, the memory 116 may store one or more software applications. Software applications stored in the memory 116 may include, for example, an operating system 143 for common computer systems as well as for software-controlled devices. Further, the memory 116 may store an entire software application, or only a part of a software application, that are executable by the processor 114. For example, the memory device 116 may store one or more radiation therapy treatment plans 142.

The data processing device 112 can communicate with the network 120 via the communication interface 118, which can be communicatively coupled to the processor 114 and the memory 116. The communication interface 118 may provide communication connections between the data processing device 112 and radiotherapy system 100 components (e.g., permitting the exchange of data with external devices). For instance, the communication interface 118 may in some embodiments have appropriate interfacing circuitry to connect to the user interface 136, which may be a hardware keyboard, a keypad, or a touch screen through which a user may input information into radiotherapy system 100.

Communication interface 118 may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as a WiFi adaptor), a telecommunication adaptor (e.g., 3G, 4G/LTE and the like), and the like Communication interface 118 may include one or more digital and/or analog communication devices that permit data processing device 112 to communicate with other machines and devices, such as remotely located components, via the network 120.

The network 120 may provide the functionality of a local area network (LAN), a wireless network, a cloud computing environment (e.g., software as a service, platform as a service, infrastructure as a service, etc.), a client-server, a wide area network (WAN), and the like. For example, network 120 may be a LAN or a WAN that may include other systems S1 (138), S2 (140), and S3 (141). Systems S1, S2, and S3 may be identical to data processing device 112 or may be different systems. In some embodiments, one or more of systems in network 120 may form a distributed computing/simulation environment that collaboratively performs the embodiments described herein. In some embodiments, one or more systems S1, S2, and S3 may include a CT scanner that obtains CT images (e.g., medical images 146). In addition, network 120 may be connected to Internet 122 to communicate with servers and clients that reside remotely on the internet.

Therefore, network 120 can allow data transmission between the data processing device 112 and a number of various other systems and devices, such as the OIS 128, the radiation therapy device 130, and the image acquisition device 132. Further, data generated by the OIS 128 and/or the image acquisition device 132 may be stored in the memory 116, the database 124, and/or the hospital database 126. The data may be transmitted/received via network 120, through communication interface 118 in order to be accessed by the processor 114, as required.

The data processing device 112 may communicate with the database 124 through network 120 to send/receive a plurality of various types of data stored on database 124. For example, the database 124 may store machine data associated with a radiation therapy device 130, image acquisition device 132, or other machines relevant to radiotherapy. The machine data information may include control points, such as radiation beam size, are placement, beam on and off time duration, machine parameters, segments, MLC configuration, gantry speed, MRI pulse sequence, and the like. The database 124 may be a storage device and may be equipped with appropriate database administration software programs. One skilled in the art would appreciate that database 124 may include a plurality of devices located either in a central or a distributed manner.

In some embodiments, the database 124 may include a processor-readable storage medium (not shown). While the processor-readable storage medium in an embodiment may be a single medium, the term “processor-readable storage medium” should be taken to include a single medium or multiple media (e.g., a centralized or distributed database, and/or associated caches and servers) that store the one or more sets of computer executable instructions or data. The term “processor-readable storage medium” shall also be taken to include any medium that is capable of storing or encoding a set of instructions for execution by a processor and that cause the processor to perform any one or more of the methodologies of the present disclosure. The term “processor readable storage medium” shall accordingly be taken to include, but not be limited to, solid-state memories, optical and magnetic media. For example, the processor readable storage medium can be one or more volatile, non-transitory, or non-volatile tangible computer-readable media.

The data processor 114 may communicate with the database 124 to read images into the memory 116, or store images from the memory 116 to the database 124. For example, the database 124 may be configured to store a plurality of images (e.g., 3D MRI, 4D MRI, 2D MRI slice images, CT images, 2D Fluoroscopy images, X-ray images, raw data from MR scans or CT scans, Digital Imaging and Communications in Medicine (DICOM) data, projection images, graphical aperture images, etc.) that the database 124 received from image acquisition device 132. Database 124 may store data to be used by the data processor 114 when executing software program 144, or when creating radiation therapy treatment plans 142. Database 124 may store the data produced by the trained machine leaning mode, such as a neural network including the network parameters constituting the model learned by the network and the resulting predicted data. The data processing device 112 may receive the imaging data, such as a medical image 146 (e.g., 2D MRI slice images, CT images, 2D Fluoroscopy images, X-ray images, 3DMR images, 4D MR images, projection images, graphical aperture images, etc.) either from the database 124, the radiation therapy device 130 (e.g., an MR-linac), and or the image acquisition device 132 to generate a treatment plan 142.

In an embodiment, the radiotherapy system 100 can include an image acquisition device 132 that can acquire medical images (e.g., MR images, 3D MRI, 2D streaming MRI, 4D volumetric MRI, CT images, cone-Beam CT. PET images, functional MR images (e.g., fMRI, DCE-MRI and diffusion MRI), X-ray images, fluoroscopic image, ultrasound images, radiotherapy portal images, SPECT images, and the like) of the patient. Image acquisition device 132 may, for example, be an MRI imaging device, a CT imaging device, a PET imaging device, an ultrasound device, a fluoroscopic device, a SPECT imaging device, or any other suitable medical imaging device for obtaining one or more medical images of the patient. Images acquired by the image acquisition device 132 can be stored within database 124 as either imaging data and/or test data. By way of example, the images acquired by the image acquisition device 132 can be also stored by the data processing device 112, as medical image 146 in memory 116.

In an embodiment, for example, the image acquisition device 132 may be integrated with the radiation therapy device 130 as a single apparatus. For example, a MR imaging device can be combined with a linear accelerator to form a system referred to as an “MR-linac.” Such an MR-linac can be used, for example, to determine a location of a target organ or a target tumor in the patient, so as to direct radiation therapy accurately according to the radiation therapy treatment plan 142 to a predetermined target.

The image acquisition device 132 can be configured to acquire one or more images of the patient's anatomy for a region of interest (e.g., a target organ, a target tumor, or both). Each image, typically a 2D image or slice, can include one or more parameters (e.g., a 2D slice thickness, an orientation, and a location, etc.) In an embodiment, the image acquisition device 132 can acquire a 2D slice in any orientation. For example, an orientation of the 2D slice can include a sagittal orientation, a coronal orientation, or an axial orientation. The processor 114 can adjust one or more parameters, such as the thickness and/or orientation of the 2D slice, to include the target organ and/or target tumor. In an embodiment, 2D slices can be determined from information such as a 3D MRI volume. Such 2D slices can be acquired by the image acquisition device 132 in “real-time” while a patient is undergoing radiation therapy treatment, for example, when using the radiation therapy device 130, with “real-time” meaning acquiring the data in at least milliseconds or less.

The data processing device 112 may generate and store radiation therapy treatment plans 142 for one or more patients. The radiation therapy treatment plans 142 may provide information about a particular radiation dose to be applied to each patient. The radiation therapy treatment plans 142 may also include other radiotherapy information, such as control points including beam angles, gantry angles, beam intensity, dose-histogram-volume information, number of radiation beams used during therapy, dose per beam, and the like.

The data processor 114 may generate the radiation therapy treatment plan 142 by using software programs 144 such as treatment planning software (e.g., Monaco®, manufactured by Elekta AB of Sweden). In order to generate the radiation therapy treatment plans 142, the data processor 114 may communicate with the image acquisition device 132 (e.g., a CT device, an MRI device, a PET device, an X-ray device, an ultrasound device, etc.) to access images of the patient and to delineate a target, such as a tumor. In some embodiments, the delineation of one or more OARs, such as healthy tissue surrounding the tumor or in close proximity to the tumor may be required. Therefore, segmentation of the OAR may be performed when the OAR is close to the target tumor. In addition, if the target tumor is close to the OAR (e.g., prostate in near proximity to the bladder and rectum), then by segmenting the OAR from the tumor, the radiotherapy system 100 may study the dose distribution not only in the target but also in the OAR.

In order to delineate a target organ or a target tumor from the OAR, medical images, such as MR images, CT images, PET images, fMR images, X-ray images, ultrasound images, radiotherapy portal images, SPECT images, and the like, of the patient undergoing radiotherapy may be obtained non-invasively by the image acquisition device 132 to reveal the internal structure of a body part. Based on the information from the medical images, a 3D structure of the relevant anatomical portion may be obtained. In addition, during a treatment planning process, many parameters may be taken into consideration to achieve a balance between efficient treatment of the target tumor (e.g., such that the target tumor receives enough radiation dose for an effective therapy) and low irradiation of the OAR(s) (e.g., the OAR(s) receives as low a radiation dose as possible). Other parameters that may be considered include the location of the target organ and the target tumor, the location of the OAR, and the movement of the target in relation to the OAR. For example, the 3D structure may be obtained by contouring the target or contouring the OAR within each 2D layer or slice of an MRI or CT image and combining the contour of each 2D layer or slice. The contour may be generated manually (e.g., by a physician, dosimetrist, or health care worker using a program such as Monaco® manufactured by Elekta AB of Sweden) or automatically (e.g., using a program such as the Atlas-based auto-segmentation software, ABAS™, manufactured by Elekta AB of Sweden). In certain embodiments, the 3D structure of a target tumor or an OAR may be generated automatically by the treatment planning software.

After the target tumor and the OAR(s) have been located and delineated, a dosimetrist, physician, or healthcare worker may determine a dose of radiation to be applied to the target tumor, as well as any maximum amounts of dose that may be received by the OAR proximate to the tumor (e.g., left and right parotid, optic nerves, eyes, lens, inner ears, spinal cord, brain stem, and the like). After the radiation dose is determined for each anatomical structure (e.g., target tumor, OAR), a process known as inverse planning may be performed to determine one or more treatment plan parameters that would achieve the desired radiation dose distribution. Examples of treatment plan parameters include volume delineation parameters (e.g, which define target volumes, contour sensitive structures, etc.), margins around the target tumor and OARs, beam angle selection, collimator settings, and beam-on times. During the inverse-planning process, the physician may define dose constraint parameters that set bounds on how much radiation an OAR may receive (e.g., defining full dose to the tumor target and zero dose to any OAR; defining 95% of dose to the target tumor; defining that the spinal cord, brain stein, and optic structures receive ≤45 Gy, ≤55 Gy and <54 Gy, respectively). The result of inverse planning may constitute a radiation therapy treatment plan 142 that may be stored in memory 116 or database 124. Some of these treatment parameters may be correlated. For example, tuning one parameter (e.g., weights for different objectives, such as increasing the dose to the target tumor) in an attempt to change the treatment plan may affect at least one other parameter, which in turn may result in the development of a different treatment plan. Thus, the data processing device 112 can generate a tailored radiation therapy treatment plan 142 having these parameters in order for the radiation therapy device 130 to provide radiotherapy treatment to the patient.

In addition, the radiotherapy system 100 may include a display device 134 and a user interface 136. The display device 134 may include one or more display screens that display medical images, interface information, treatment planning parameters (e.g., projection images, graphical aperture images, contours, dosages, beam angles, etc.) treatment plans, a target, localizing a target and/or tracking a target, or any related information to the user. The user interface 136 may be a keyboard, a keypad, a touch screen or any type of device that a user may input information to radiotherapy system 100. Alternatively, the display device 134 and the user interface 136 may be integrated into a device such as a tablet computer (e.g., Apple iPad®, Lenovo Thinkpad®, Samsung Galaxy®, etc.).

Furthermore, any and all components of the radiotherapy system 100 may be implemented as a virtual machine (e.g., VMWare, Hyper-V, and the like). For instance, a virtual machine can be software that functions as hardware. Therefore, a virtual machine can include at least one or more virtual processors, one or more virtual memories, and one or more virtual communication interfaces that together function as hardware. For example, the data processing device 112, the OIS 128, the image acquisition device 132 could be implemented as a virtual machine. Given the processing power, memory, and computational capability available, the entire radiotherapy system 100 could be implemented as a virtual machine.

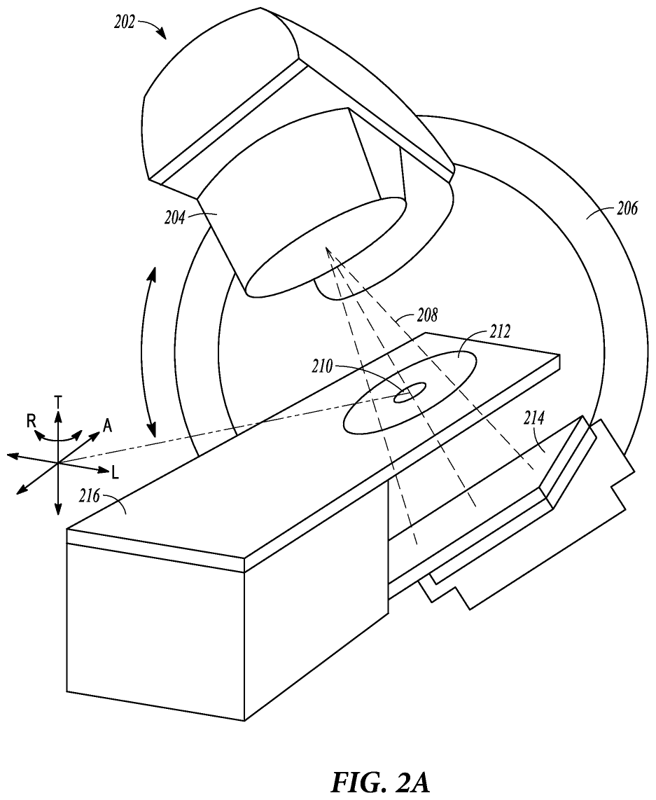

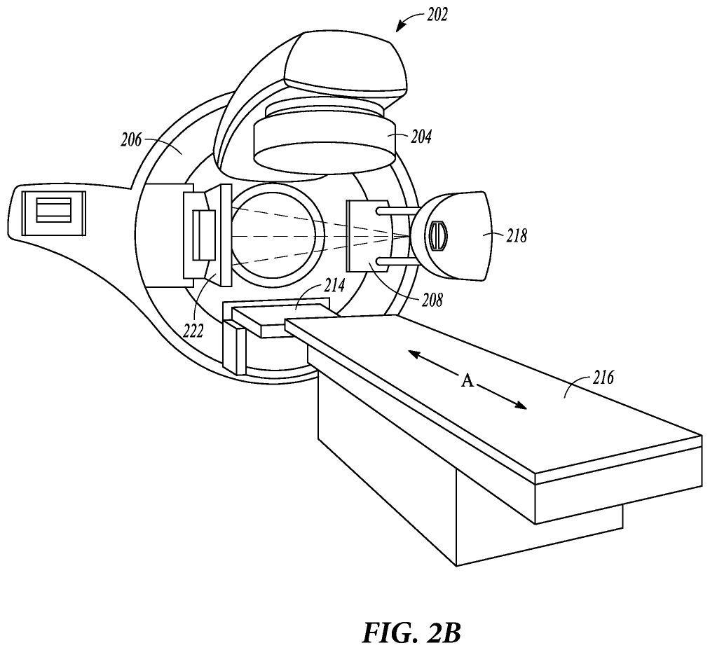

FIG. 2A illustrates an exemplary radiation therapy device 202 that may include a radiation source (e.g, an X-ray source or a linac), a couch 216, an imaging detector 214, and a radiation therapy output 204. The radiation therapy device 202 may be configured to emit a radiation beam 208 to provide therapy to a patient. The radiation therapy output 204 can include one or more attenuators or collimators, such as an MLC. A patient can be positioned in a region 212 and supported by the couch 216 to receive a radiation therapy dose, according to a radiation therapy treatment plan. The radiation therapy output 204 can be mounted or attached to a gantry 206 or other mechanical support. One or more chassis motors (not shown) may rotate the gantry 206 and the radiation therapy output 204 around the couch 216 when the couch 216 is inserted into the treatment area. In an embodiment, the gantry 206 may be continuously rotatable around the couch 216 when the couch 216 is inserted into the treatment area. In another embodiment, the gantry 206 may rotate to a predetermined position when the couch 216 is inserted into the treatment area. For example, the gantry 206 can be configured to rotate the therapy output 204 around an axis (“A”). Both the couch 216 and the radiation therapy output 204 can be independently moveable to other positions around the patient, such as moveable in transverse direction (“T”), moveable in a lateral direction (“L”), or as rotation about one or more other axes, such as rotation about a transverse axis (indicated as “R”). A controller communicatively connected to one or more actuators (not shown) may control the couch 216 movements or rotations in order to properly position the patient in or out of the radiation beam 208 according to a radiation therapy treatment plan. Both the couch 216 and the gantry 206 are independently moveable from one another in multiple degrees of freedom, which allows the patient to be positioned such that the radiation beam 208 can target the tumor. The MLC may be integrated with the gantry 206 to deliver the radiation beam 208 of a certain shape.

The coordinate system (including axes A, T, and L) shown in FIG. 2A can have an origin located at an isocenter 210. The isocenter can be defined as a location where the central axis of the radiation beam 208 intersects the origin of a coordinate axis, such as to deliver a prescribed radiation dose to a location on or within a patient. Alternatively, the isocenter 210 can be defined as a location where the central axis of the radiation beam 208 intersects the patient for various rotational positions of the radiation therapy output 204 as positioned by the gantry 206 around the axis A. As discussed herein, the gantry angle corresponds to the position of gantry 206 relative to axis A, although any other axis or combination of axes can be referenced and used to determine the gantry angle.

The gantry 206 may have an attached imaging detector 214 that is preferably opposite the radiation therapy output 204. In an embodiment, the imaging detector 214 can be located within a field of the therapy beam 208. The imaging detector 214 can maintain alignment with the therapy beam 208. The imaging detector 214 can rotate about the rotational axis as the gantry 206 rotates. In an embodiment, the imaging detector 214 can be a flat panel detector (e.g., a direct detector or a scintillator detector). In this manner, the imaging detector 214 can be used to monitor the therapy beam 208 or the imaging detector 214 can be used for imaging the patient's anatomy, such as portal imaging. The control circuitry of radiotherapy device 202 may be integrated within system 100 or remote from it.

In an illustrative embodiment, one or more of the couch 216, the therapy output 204, or the gantry 206 can be automatically positioned, and the therapy output 204 can establish the therapy beam 208 according to a specified dose for a particular therapy delivery instance. A sequence of therapy deliveries can be specified according to a radiation therapy treatment plan, such as using one or more different orientations or locations of the gantry 206, the couch 216, or the therapy output 204. The therapy deliveries can occur sequentially, but can intersect in a desired therapy locus on or within the patient, such as at the isocenter 210. A prescribed dose of radiation therapy can thereby be delivered to the therapy locus while damage to tissue near the therapy locus can be reduced or avoided.

FIG. 2B illustrates an exemplary radiotherapy system 202 that combines a radiation system (e.g., a linac) and a CT imaging system. The radiation therapy device 202 can include an MLC (not shown). The CT imaging system can include an imaging X-ray source 218, such as providing X-ray energy in a kiloelectron-Volt (keV) energy range. The imaging X-ray source 218 can provide a fan-shaped and/or a conical beam 208 directed to an imaging detector 222, such as a flat panel detector. The radiation therapy device 202 can be similar to the system described in relation to FIG. 2A, such as including a radiation therapy output 204, a gantry 206, a couch 216, and another imaging detector 214 (such as a flat panel detector). The X-ray source 218 can provide a comparatively-lower-energy X-ray diagnostic beam, for imaging.

As illustrated in FIG. 2B, the radiation therapy output 204 and the X-ray source 218 can be mounted on the same rotating gantry 206, rotationally-separated from each other by 90 degrees. In some examples, two or more X-ray sources can be mounted along the circumference of the gantry 206, such that each has its own detector arrangement to provide multiple angles of diagnostic imaging concurrently. Similarly, multiple radiation therapy outputs 204 can be provided.

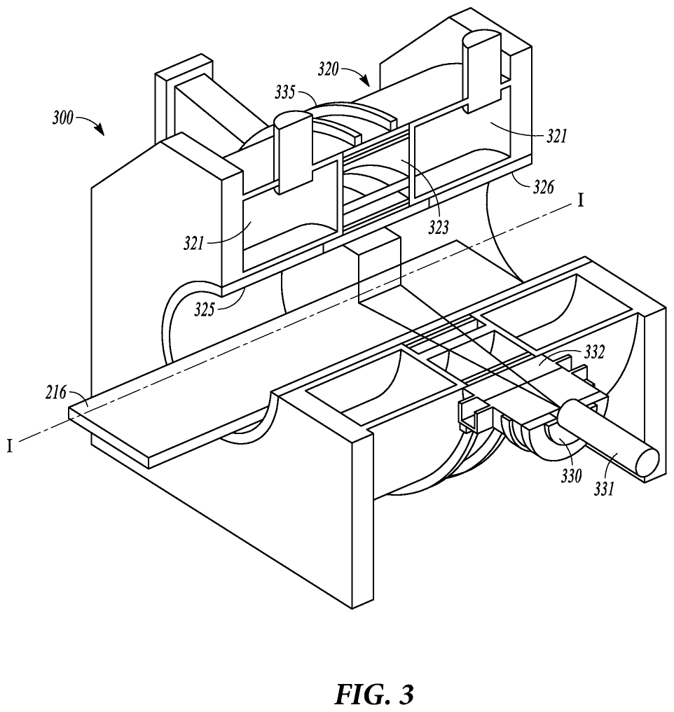

FIG. 3 illustrates an exemplary radiotherapy system 300 that combines a radiation system (e.g., a linac) and a nuclear MR imaging system, also referred to as an MR-linac system. The system 300 may include a couch 216, an image acquisition device 320, and a radiation delivery device 330. The system 300 can deliver radiation therapy to a patient in accordance with a radiotherapy treatment plan, such as the treatment plan 142 created and stored in the memory 116. In some embodiments, the image acquisition device 320 may correspond to the image acquisition device 132 in FIG. 1 that may acquire images of a first modality (e.g., an MR image) or destination images of a second modality (e.g, a CT image).

The couch 216 may support a patient during a treatment session. In some implementations, the couch 216 may move along a horizontal translation axis (labelled “I”), such that the couch 216 can move the patient resting on the couch 216 into and/or out of the system 300. The couch 216 may also rotate around a central vertical axis of rotation, transverse to the translation axis. To allow such movement or rotation, the couch 216 may have motors (not shown) enabling the couch to move in various directions and to rotate along various axes. A controller (not shown) may control these movements or rotations in order to properly position the patient according to a treatment plan.

In some embodiments, the image acquisition device 320 may include an MR imaging machine that can acquire 2D or 3D MR images of the patient before, during, and/or after a treatment session. The image acquisition device 320 may include a magnet 321 for generating a primary magnetic field for magnetic resonance imaging. The magnetic field lines generated by operation of the magnet 321 may run substantially parallel to the central translation axis “I”. The magnet 321 may include one or more coils with an axis that runs parallel to the translation axis “I”. In some embodiments, the one or more coils in magnet 321 may be spaced such that a central window 323 of magnet 321 is free of coils. In other embodiments, the coils in magnet 321 may be thin enough or of a reduced density such that they are substantially transparent to radiation of the wavelength generated by radiotherapy device 330. In some embodiments, the image acquisition device 320 may also include one or more shielding coils, which may generate a magnetic field outside the magnet 321 of approximately equal magnitude and opposite polarity in order to cancel or reduce any magnetic field outside of the magnet 321. As described below, a radiation source 331 of radiotherapy device 330 may be positioned in the region where the magnetic field is cancelled, at least to a first order, or reduced.

The image acquisition device 320 may also include two gradient coils 325 and 326, which may generate a gradient magnetic field that is superposed on the primary magnetic field. The coils 325 and 326 may generate a gradient in the resultant magnetic field that allows spatial encoding of the protons so that their position can be determined. The gradient coils 325 and 326 may be positioned around a common central axis with the magnet 321 and may be displaced along that central axis. The displacement may create a gap, or window, between the coils 325 and 326. In embodiments where the magnet 321 includes a central window 323 between the coils, the two windows may be aligned with each other.

In some embodiments, the image acquisition device 320 may be an imaging device other than an MRI, such as an X-ray, a CT, a CBCT, a spiral CT, a PET, a SPECT, an optical tomography, a fluorescence imaging, ultrasound imaging, radiotherapy portal imaging device, or the like. As would be recognized by one of ordinary skill in the art, the above description of image acquisition device 320 concerns certain embodiments and is not intended to be limiting.

The radiotherapy device 330 may include the radiation source 331 (e.g., an X-ray source or a linac), and a collimator such as an MLC 332. A collimator is a beam-limiting device that can help to shape the beam of radiation emerging from the machine and can limit the maximum field size of a beam. The MLC 332 can be used for shaping, directing, or modulating an intensity of a radiation therapy beam to the specified target locus within the patient. The MLC 332 can include metal collimator plates, also known as MLC leaves, which slide into place to form the desired field shape. The radiotherapy device 330 may be mounted on a chassis 335. One or more chassis motors (not shown) may rotate chassis 335 around the couch 216 when the couch 216 is inserted into the treatment area. In an embodiment, chassis 335 may be continuously rotatable around the couch 216, when the couch 216 is inserted into the treatment area. The chassis 335 may also have an attached radiation detector (not shown), preferably located opposite to radiation source 331 and with the rotational axis of chassis 335 positioned between radiation source 331 and the detector. Further, device 330 may include control circuitry (not shown) used to control, for example, one or more of the couch 216, image acquisition device 320, and radiotherapy device 330. The control circuitry of radiotherapy device 330 may be integrated within system 300 or remote from it.

During a radiotherapy treatment session, a patient may be positioned on the couch 216. System 300 may then move the couch 216 into the treatment area defined by magnetic 321 and coils 325, 326, and chassis 335. Control circuitry may then control the radiation source 331, MLC 332, and the chassis motor(s) to deliver radiation to the patient through the window between coils 325 and 326 according to a radiotherapy treatment plan.

The radiation therapy output configurations illustrated in FIGS. 2A-2B and 3, such as the configurations where a radiation therapy output can be rotated around a central axis (e.g., an axis “A”), are for the purpose of illustration and not limitation Other radiation therapy output configurations can be used. For example, a radiation therapy output can be mounted to a robotic arm or manipulator having multiple degrees of freedom. In yet another embodiment, the therapy output can be fixed, such as located in a region laterally separated from the patient, and a platform supporting the patient can be used to align a radiation therapy isocenter with a specified target locus within the patient.

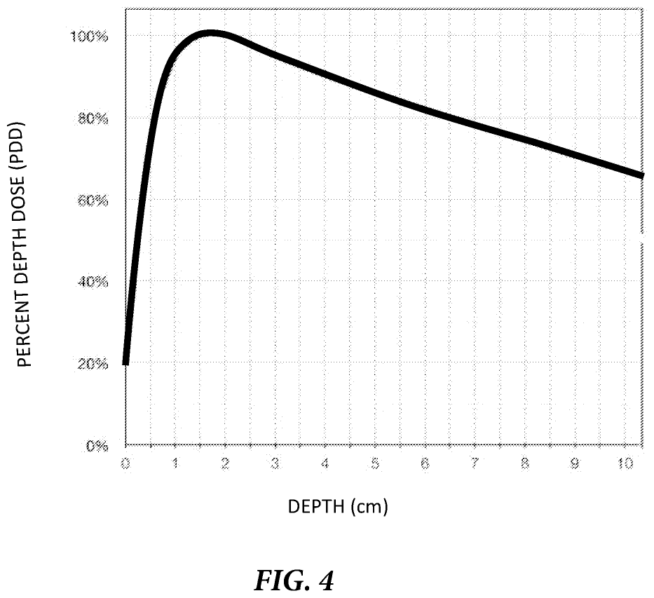

FIG. 4 illustrates an example of a dose profile. The dose profile represents an algorithm-generated spatial distribution of radiation dose, such as generated by a radiation treatment planning software in the software programs 144. Examples of dose profile can include a percent depth dose (PDD) profile representing changes of relative dose with depth, or a percentile radial dose (PRD) profile representing changes of relative dose with a radial distance. The PDD, as illustrated in FIG. 4, can be measured by applying radiation from a radiation machine to a medium (e.g., a phantom) that simulates human tissue. In an example, the phantom can include water. The amount of radiation deposited, as a function of depth in the medium, can be measured using sensors installed in the medium and recorded, such as to provide the PDD curve shown in FIG. 4. In addition to PDD or PRD profiles, in some examples, the dose profile may include beam model parameter values. Examples of the beam model parameters can include a size and position of one or more photon sources within the radiation machine, a maximum energy of a photon spectrum for photons emitted from the radiation machine, a number of factors describing the shape of a photon spectrum emitted from the radiation machine, a size and position of one or more electron sources within the radiation machine, an average energy of an electron spectrum emitted from the radiation machine, or one or more numbers describing how radiation (e.g., electrons or photons) emitted by the radiation machine can vary off-axis, among others.

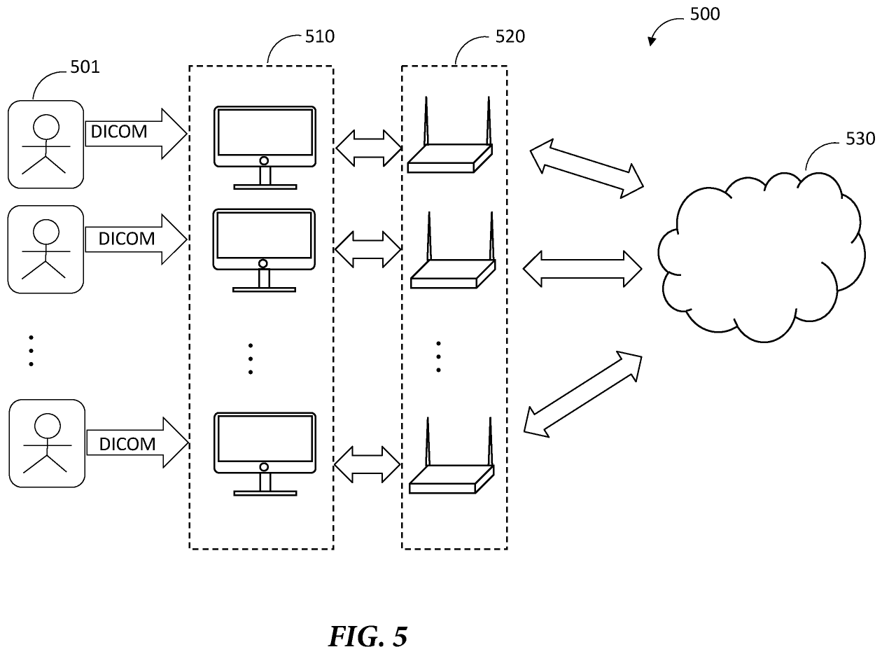

FIG. 5 is a diagram illustrating an exemplary architecture of a cloud-based dose verification system 500. The system 500 can be configured to determine a secondary radiation dose profile, and to use the secondary dose profile to verify a primary dose profile generated by a TPS of a radiotherapy system, such as the system 202 or the system 300. The secondary dose profile can be determined independently from the TPS that determines the primary radiation dose profile. The system 500 may include multiple access points for multiple clients 510, one or more gateways 520, and a cloud-based computing device or networked devices 530 (also referred to as a cloud server, or simply “cloud”) configured to provide a suite of cloud-based services including dose evaluation and verification.

The one or more clients 510 (or “tenants”) are devices that can be located in different hospitals or clinical facilities. Examples of the clients 510 may include computers, tablets, or mobile devices (e.g., mobile phones), among others. A client can subscribe one or more cloud services provided by the cloud 530, and be securely connected to the cloud 530 over an Internet connection (e.g., Ethernet, or wireless connection such as WiFi or a cellular network) Image information acquired from a patient 501, such as produced by a radiotherapy system (e.g., an MR-linac) in a hospital or clinical facility, can be input to the client 510. The patient image data may include a digital image of a radiotherapy target of the subject, which may include a computed tomography (CT) image, a synthetic CT image, or a magnetic resonance (MR) image, among others. Machine information, such as one or more machine parameters (also referred to as “control points”) of the radiation machine that produce said patient image information, may also be provided to the client 510. Examples of the machine parameters may include linac gantry angles, beam aperture shapes through which the therapeutic radiation beams are projected at the target, and the beams' intensities, or one or more parameters associated with delivery electron or particle therapy, among others.

In an example, patient image information and machine information can be formatted into a patient file according to a particular standard, such as digital imaging and communications in medicine (DICOM) standard. Such a patient file is referred to as a DICOM file in this document. A DICOM file stores metadata including patient image information and machine parameters. The patient image information may include image type, image pixel data, image position, image orientation, image plane pixel spacing, pixel aspect ratio, pixel location, photometric interpretation. In an example, a DICOM file may store a set of images taken at different gantry angles, such that the radiation beam is delivered from different directions to the target tumor and OARs. The machine parameters include parameters characterizing configuration and operation status (the “control points”) of the radiation machine and treatment plan using said machine, such as beam number, beam name, beam description, beam type, beam intensity, beam limiting device type, gantry angle, gantry rotation direction, leaf/jaw positions, aperture weight or intensity, beam angle selection, collimator settings, or beam-on times, among others. A DICOM file may additionally include structure information such as anatomical structures of interest in a patient, volume delineation parameters that define target volumes, contour sensitive structures, etc., or margins around the target tumor and OARs, among others. The treatment plan information stored in a DICOM file may also include dose information, which may include a primary dose profile (e.g., one or more dose metrics or dose statistics).

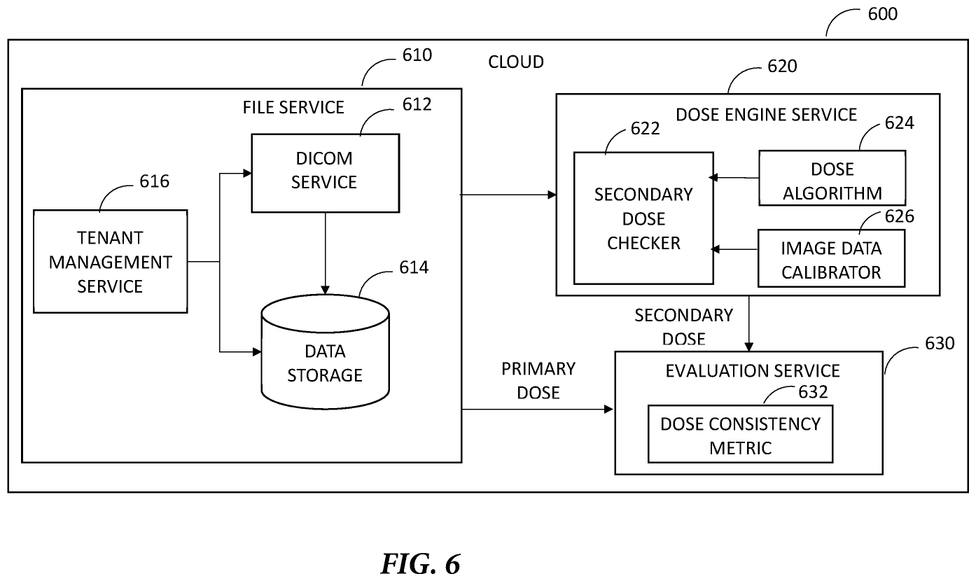

The clients 510 may establish a communication with the cloud 530, and upon authentication, upload the patient DICOM files to the cloud, such as being stored and maintained in a cloud storage. To save communication bandwidth and improve communication and storage efficiency, the DICOM files may be compressed prior to transmission and storage. The compression can be lossy or lossless compression. Examples of the compression algorithms may include predictive coding, arithmetic coding, Huffman coding, adaptive Lempel-Ziv-Welch algorithm, chain code, transform coding, fractal compression, among others. Data encryption may be performed on the patient DICOM file before data transmission and storage for confidentiality and satisfaction of compliance requirement. Additionally or alternatively, the client devices 510 can use a subscribed DICOM service, provided by the cloud 530, to extract one or more of the image information, the machine information, or the treatment plan information from the DICOM file. Examples of and cloud storage and cloud-based services such as DICOM service are discussed below such as with reference to FIG. 6.

Communication between the clients 510 and the cloud 530 may follow Transmission Control Protocol/Internet Protocol (TCP/IP), Wi-Fi Protected Access security protocol, 3G or 4G cellular network protocols, Long-Term Evolution (LTE), or other wireless network protocols. As may be appreciated by one skilled in the art, other network topologies and arrangements may be used. In an example as illustrated in FIG. 5, the clients 510 may communicate with the cloud 530 via gateways 520. The gateways 520 may include multiple devices, such as multiple routers, that form an asset group. The gateways 520 may support one or more communication protocols to control and monitor data flow between the clients 510 to the cloud 530, such as an Internet protocol via Ethernet or a wireless network (e.g., WiFi, or cellular network). In some examples, the gateways 520 may include a computer or computer programs configured to perform specific tasks, such as data aggregation, identity recognition, security, data buffering, alerting, gateway analytics, monitoring service connectivity, among others. In an example, when multiple clients simultaneously access the cloud services, the gateways 520 may monitor and control traffic between the clients 510 and the cloud 530.

The cloud 530 may include a server or networked servers configured to store and manage data uploaded by one or more clients (e.g., patient DICOM files, or parsed DICOM data), run applications such as secondary dose check and dose verification against primary dose profiles generated by different TPS systems, or deliver content (e.g., verified dose profiles) to one or more subscribed clients. The cloud 530 can be updated with access policies or whitelists. In some examples, the cloud 530 can be an on-premise cloud. The cloud 530 may have a scalable infrastructure that supports services such as imaging data (e.g., DICOM files) storage and processing. Examples of the cloud 530 and various services hosted therein are discussed below, such as with reference to FIG. 6.

A system user (e.g., a radiation oncologist, a medical physicist, or a healthcare professional) may use a client 510 to access the cloud 130. The client 510 may be provided with a web browser for accessing a web server via Hypertext Transfer Protocol (HTTP) or an encrypted communication protocol (e.g., Hypertext Transfer Protocol for secure communication (HTTPS)). In an example, upon necessary user authentication (e.g., account ID, password, bearer token etc.), a user may query the cloud 130 (or a portion thereof, such as the patient database or the services) for information such as secondary dose profiles, dose verification results, among others. The client 510 may include applications, software programs, or visualization tools that present the secondary dose check and dose verification results to a user, such as to display on a screen of the user interface. The information may be presented in a table, a chart, a diagram, or an interactive dashboard, and can include textual and graphical contents. Hard copies of such information may be printed. The information presented to the user may also include machine information and patient image information extracted from patient DICOM files.

The client 510 may generate an alert notification to alert the user 501 about an unverified dose profile, such as an inconsistency between the primary dose profile produced by a TPS of a radiotherapy system and the secondary dose profile independently calculated by a dose engine application in the cloud 530. The alert notification may be sent via email, text or “Instant” messaging such as short message service (SMS), Web page update, phone or pager call, among others. In some examples, alert notification is triggered only when a specific alert condition is satisfied. Upon alert notification, the user may view the alert, interpret the results (e.g., secondary dose profile), and take actions such as performing further dose testing (automatic or manual dose check), or making adjustment to the dose engine application in the cloud 530.

Portions of the dose verification system 500 may be implemented using hardware, software, firmware, or combinations thereof. In an example, at least a portion of the dose verification system 500 may be implemented using an application-specific circuit that may be constructed or configured to perform one or more particular functions, or may be implemented using a general-purpose circuit that may be programmed or otherwise configured to perform one or more particular functions. Such a general-purpose circuit may include a microprocessor or a portion thereof, a microcontroller or a portion thereof, or a programmable logic circuit, a memory circuit, a network interface, and various components for interconnecting these components. For example, a “comparator” may include, among other things, an electronic circuit comparator that may be constructed to perform the specific function of a comparison between two signals or the comparator may be implemented as a portion of a general-purpose circuit that may be driven by a code instructing a portion of the general-purpose circuit to perform a comparison between the two signals.

FIG. 6 is a block diagram illustrating an exemplary cloud server 600 configured to provide data storage and a suite of cloud-based services including dose verification. The cloud server 600 can be a physical or virtual infrastructure. The cloud server 600, which can be an embodiment of at least a portion of the cloud 530 shown in FIG. 5, can include a file service 610, a dose engine service 620, and a dose evaluation service 630. The file service 610 manages patient files received from one or more clients 510. Each patient file may contain patient image information and information about the radiation machine. In an example, the patient file includes a patient DICOM file. The file service 610 may include a DICOM service 612 configured to parse a DICOM file using a parser application and extract various types of information therefrom, including one or more of patient image information, machine information, dose information, structure information, and treatment plan information, as discussed above with reference to FIG. 5. The file service 610 may include a data storage 614 to store the patient DICOM files received from multiple tenants 510, or parsed DICOM information.

In some examples, the DICOM files received from the clients 510 and stored in the data storage 614 are compressed and/or encrypted DICOM files. The DICOM service may include applications to decompress and/or decrypt a stored DICOM file before parsing and extracting information from the DICOM file.

The file service 610 may include a tenant management service 616 configured to manage service requests from multiple clients 510. The tenant management service 616 can dynamically allocate physical and virtual resources to process service requests from one or more clients. In an example, the tenant management service 616 can queue clients and their service requests based on the computing and storage resources at the cloud server 600, amount of data requested to be processed by each client, type of services requested, quality-of-service (QoS) requirement imposed by each client, among others. Dynamic allocation of physical or virtual resources may include distributed computing among multiple networked computing devices that collaboratively accomplish one particular computing task, such as executing one or more services of DICOM file parsing, secondary dose check, or dose verification for a DICOM file provided by a client. Additionally or alternatively, dynamic assignment of system resources can include parallel computing among multiple processors (such as in a cloud server) that simultaneously execute multiple tasks for different clients, such as services of DICOM file parsing, secondary dose check, or dose verification, associated with DICOM files uploaded by different clients. The dynamic allocation of physical or virtual resources, including distributed computing and/or parallel computing, can help to improve the efficiency of multi-tenant management and the quality of service to meet the demands of different clients.

The dose engine service 620 can include a secondary dose checker 622 configured to generate a secondary dose profile using parsed DICOM information (e.g., one or more of patient image information, machine parameters, or treatment plan parameters). In an example, patient images uploaded to and stored in the cloud storage include gray-scale CT or MR images. The dose engine service 620 can retrieve a gray-scale image, and an image data calibrator 626 can calibrate the gray-scale image by converting the pixel data to mass density data. For example, the image data calibrator 626 can convert the CT numbers in a CT image (which represent X-ray absorption coefficient of the image pixels, expressed in Hounsfield units) into relative electron density (ED) data or mass density (MD) data using a pre-generated look-up table or formula. The converted ED or MD data of the patient image can then be used by the secondary dose checker 622 to generate the secondary dose profile.

The secondary dose checker 622 can calculate a secondary dose profile using a dose algorithm 624. In an example, the cloud server 600 can provide an integration interface that allows a user to upload one or more distinct dose algorithms, and the secondary dose checker 622 can calculate one or more secondary dose profiles using the respective dose algorithms.

In an example, the dose algorithm 624 can be a commercial algorithm used by a radiotherapy system, such as Monaco® (Elekta systems AB). Implementation of such a commercial algorithm in the dose engine service 620 can be different from their corresponding commercial algorithm implementation. In some examples, the dose algorithm 624 can be different from the commercial algorithm used by a radiotherapy system.

The dose algorithm 624 (hereinafter referred to as a “secondary dose algorithm”) can be different from the algorithm used by the radiation machine (hereinafter referred to as a “primary dose algorithm”) to calculate the primary dose profile. The secondary dose profile thus generated may not be identical to the primary dose profile, and therefore can be used to verify the accuracy of the primary dose profile. In an example, the secondary algorithm can be a more sophisticated algorithm than the primary dose algorithm. In another example, the primary and secondary dose algorithms are distinct implementations of the same dose calculation methodology, such as implementations done by different manufacturers or vendors. By way of example and not limitation, the primary dose algorithm is a pencil beam algorithm implemented in the TPS of a radiotherapy system, and a user can choose from implemented algorithms, or upload and integrate into the cloud, a Monte Carlo algorithm, which can more accurately simulate particle movement, can be used as the secondary dose algorithm for dose verification. Alternatively, the secondary dose algorithm can be a different implementation of pencil beam algorithm than the implementation in the TPS of the radiotherapy system.

In an example, information about the identified primary dose algorithm can be presented to a user, such as via a user interface of the client device 510. The user is prompted to upload the secondary dose algorithm, which can be different from the primary dose algorithm, or a different implementation of the primary dose algorithm. In some examples, the dose algorithm 624 can include implementations of a plurality of candidate dose algorithms. A user can choose, via the user interface, a secondary dose algorithm from the plurality of candidate dose algorithms. If none of the candidate algorithms is chosen, the user can upload a secondary dose algorithm and integrate it into the dose algorithm 624.

The dose evaluation service 630 can compare the secondary dose profile, such as generated by the dose engine service 620, with the primary dose profile. The dose evaluation service 630 can generate a metric of consistency 632 between the primary and secondary dose profiles. In an example, the dose consistency metric 632 can include a relative difference between a primary dose metric extracted from the patient DICOM file and a secondary dose metric generated by the secondary dose checker 622. The primary and secondary dose metrics can be of the same type. Examples of the dose metric can include one or more of a maximum dose, a minimum dose, a dose range, a coverage region (e.g., contour of coverage), a 3D dose distribution, a dose volume histogram (DVH), or an overlap volume histogram (OVH), among others. The evaluation service 630 may determine that the primary dose profile is verified if the dose consistency metric 632 satisfies a specific condition, such as falling below a threshold or within a specified range. The verified dose profile can be used to generate a treatment plan, such as by using a beam model stored in the cloud server 600. In an example, the cloud server 600 can provide an integration interface that allows a user to upload a beam model, and apply the beam model to the verified dose profile to generate a treatment plan.

The dose verification results, optionally the secondary dose check results such as secondary dose profiles, the treatment plan generated based on the verified dose profiles, among other computation results generated by the cloud services, can be accessed by authenticated clients 510 through the communication link shown in FIG. 5, and presented to a user such as displayed on a screen of the user interface.

In various examples, the cloud server 600 may include multiple networked computing devices that work collaboratively to fulfill client requests for various cloud services, such as parsing the DICOM files, generating the secondary dose profile, or generating dose verification results. In an example, a network of multiple computers can, via parallel computing, process DICOM files and verify doses simultaneously for multiple patients, or dissect a patient DICOM file and extract images corresponding to different gantry angles and verifying dose simultaneously based on the multiple images.

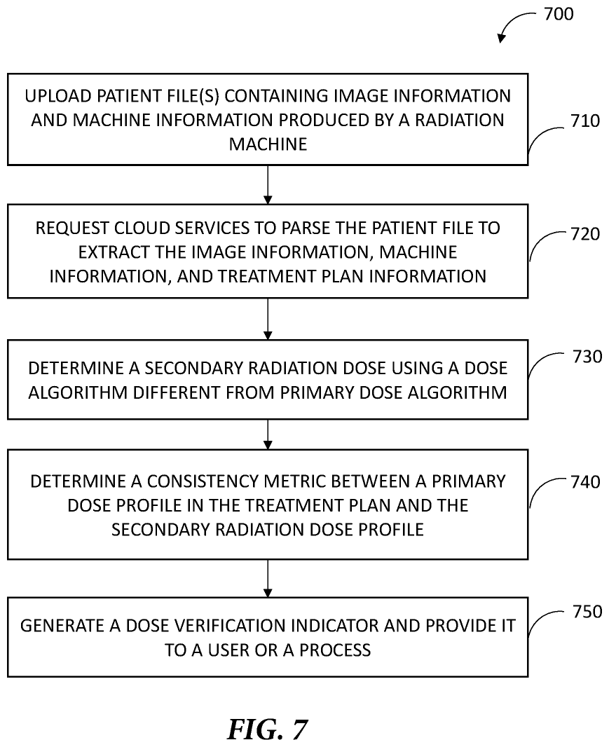

FIG. 7 is a flow-chart illustrating an exemplary method 700 of verifying a dose profile using cloud services, such as the services provided by the cloud 530 or the cloud server 600. The dose profile to be verified, also referred to as a “primary dose profile”, can be generated by a TPS system of a radiotherapy system, such as the system 202 or the system 300. In an example, the method 700 may be implemented in and executed by the dose verification system 500.

The method 700 commences at 710, where one or more patient files each containing image information and machine information produced by a radiation machine can be uploaded to cloud-based computing device or networked devices, such as the cloud 530 or 600. In an example, the patient file can include a DICOM file acquired from a patient by a radiation machine in a hospital or clinical facility. Each DICOM file stores metadata including patient image information and machine parameters. The patient image information may include a digital image of a radiotherapy target of the subject, which may include a computed tomography (CT) image, a synthetic CT image, or a magnetic resonance (MR) image, among others. The machine information include information about structure, configuration, and operation status of the radiation machine, such as one or more machine parameters (also referred to as “control points”) of the radiation machine that produce the patient image information. A DICOM file may additionally include treatment plan information, such as a primary dose profile (e.g., one or more dose metrics or dose statistics) generated by the TPS. In some examples, a DICOM file may store multiple images of a patient produced at distinct gantry angles, along with machine parameters, treatment plan parameters, and primary dose metrics and statistics.

The DICOM file can be uploaded to the cloud through a client device, such as the client 510. A client can be securely connected to the cloud over an Internet connection (e.g., Ethernet, or wireless connection such as WiFi or a cellular network), and subscribe one or more cloud services. At 720, upon necessary user authentication, a client can request cloud services (e.g., the file service 610 provided by the cloud server 600) to parse the patient file to extract the image information, machine information, and treatment plan information. In an example, multiple images corresponding to distinct gantry angles can be extracted from a DICOM file. In case that multiple clients simultaneously request cloud services, the clients and their service requests may be scheduled such as based on the computing and storage resources, amount of data requested to be processed by each client, type of services requested, quality-of-service (QoS) requirement imposed by each client, among others. In an example, the client management may include dynamically allocating physical and virtual resources to each requesting client, or using distributed computing or parallel computing to improve the efficiency of multi-tenant management and the quality of service to meet the demands of different clients.

At 730, a secondary dose profile can be determined using one or more of the patient image information, machine parameters, or treatment plan parameters, such as obtained from the parsed DICOM file. A dose algorithm (also referred to as a secondary dose algorithm) can be applied to the patient and machine information to calculate a secondary dose profile. The secondary dose algorithm, which can be uploaded by a user to the cloud via an integration interface, can be a commercial dose algorithm. Alternatively, the secondary dose algorithm can be different from commercial dose algorithms.

The secondary dose algorithm can be different from the dose algorithm used by the radiation machine to calculate the primary dose profile (also referred to as a primary dose algorithm). In an example, the primary and secondary dose algorithms are distinct implementations of the same dose calculation methodology. In an example, the user can upload the secondary dose algorithm to the cloud. In another examples, a user can choose, via a user interface, a secondary dose algorithm from a plurality of candidate dose algorithms implemented in the cloud. If none of the candidate algorithms is chosen, the user can upload a secondary dose algorithm. Examples of dose profile can include a percent depth dose (PDD) profile representing changes of relative dose with depth, or a percentile radial dose (PRD) profile representing changes of relative dose with a radial distance, beam model parameter values, a dose-volume histogram, an overlap volume histogram, or a three-dimensional dose distribution, among others.

At 740, the secondary radiation dose profile determined at 730 is compared to the primary dose profile generated by the TPS of the radiation machine, and a consistency metric between the primary and secondary dose profiles can be generated, such as using the dose evaluation service 630. In an example, the dose consistency metric can include a relative difference between a primary dose metric (obtained from the primary dose profile) and a secondary dose metric (obtained from the secondary dose profile). The primary and secondary dose metrics can be of the same type. The evaluation service may determine that the primary dose profile is verified if the dose consistency metric satisfies a specific condition, such as falling below a threshold or within a specified range.

At 750, a dose verification indicator can be generated. The dose verification results, optionally the secondary dose check results such as secondary dose profiles, among other computation results generated by the cloud services, can be accessed by authenticated clients, and presented to a user such as displayed on a screen of the user interface. If the difference between a primary dose metric and a secondary dose metric exceeds a threshold, an alert notification may be generated, such as on a client side, to alert the user of the unverified dose profile. The user may then take actions such as performing further dose testing (automatic or manual dose check), or making adjustment to the dose engine service in the cloud. The verified dose profile, along other information, may be presented to a user. In some examples, the verified dose profile can be used to generate a treatment plan, such as by using a beam model.

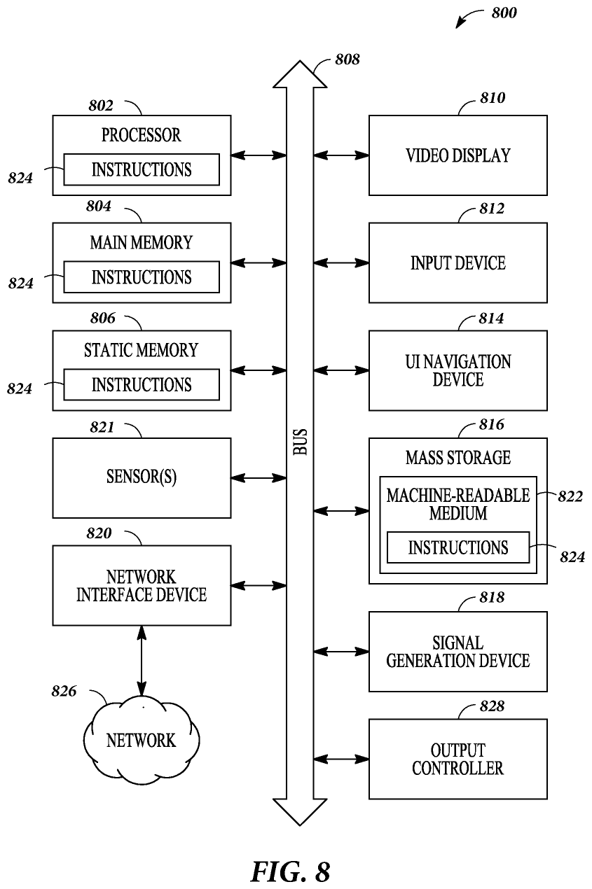

FIG. 8 illustrates a block diagram of an embodiment of a machine 800 on which one or more of the methods as discussed herein can be implemented. In one or more embodiments, one or more items of the data processing device 112 can be implemented by the machine 800. In alternative embodiments, the machine 800 operates as a standalone device or may be connected (e.g., networked) to other machines. In one or more embodiments, the data processing device 112 can include one or more of the items of the machine 800. In a networked deployment, the machine 800 may operate in the capacity of a server or a client machine in server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example machine 800 includes processing circuitry 802 (e.g., a central processing unit (CPU), a graphics processing unit (GPU), an application specific integrated circuit, circuitry, such as one or more transistors, resistors, capacitors, inductors, diodes, logic gates, multiplexers, buffers, modulators, demodulators, radios (e.g., transmit or receive radios or transceivers), sensors 821 (e.g., a transducer that converts one form of energy (e.g., light, heat, electrical, mechanical, or other energy) to another form of energy), or the like, or a combination thereof), a main memory 804 and a static memory 806, which communicate with each other via a bus 808. The machine 800 (e.g., computer system) may further include a video display unit 810 (e.g., a liquid crystal display (LCD) or a cathode ray tube (CRT)). The machine 800 also includes an alphanumeric input device 812 (e.g., a keyboard), a user interface (UI) navigation device 814 (e.g., a mouse), a disk drive or mass storage unit 816, a signal generation device 818 (e.g., a speaker) and a network interface device 820.

The disk drive unit 816 includes a machine-readable medium 822 on which is stored one or more sets of instructions and data structures (e.g., software) 824 embodying or utilized by any one or more of the methodologies or functions described herein. The instructions 824 may also reside, completely or at least partially, within the main memory 804 and/or within the processor 802 during execution thereof by the machine 800, the main memory 804 and the processor 802 also constituting machine-readable media.

The machine 800 as illustrated includes an output controller 828. The output controller 828 manages data flow to/from the machine 800. The output controller 828 is sometimes called a device controller, with software that directly interacts with the output controller 828 being called a device driver.

While the machine-readable medium 822 is shown in an embodiment to be a single medium, the term “machine-readable medium” may include a single medium or multiple media (e.g., a centralized or distributed database, and/or associated caches and servers) that store the one or more instructions or data structures. The term “machine-readable medium” shall also be taken to include any tangible medium that is capable of storing, encoding or carrying instructions for execution by the machine and that cause the machine to perform any one or more of the methodologies of the present invention, or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including by way of example semiconductor memory devices, e.g., Erasable Programmable Read-Only Memory (EPROM), Electrically Erasable Programmable Read-Only Memory (EEPROM), and flash memory devices; magnetic disks such as internal hard disks and removable disks; magneto-optical disks; and CD-ROM and DVD-ROM disks.

The instructions 824 may further be transmitted or received over a communications network 826 using a transmission medium. The instructions 824 may be transmitted using the network interface device 820 and any one of a number of well-known transfer protocols (e.g., HTTP). Examples of communication networks include a local area network (“LAN”), a wide area network (“WAN”), the Internet, mobile telephone networks, Plain Old Telephone (POTS) networks, and wireless data networks (e.g., WiFi and WiMax networks). The term “transmission medium” shall be taken to include any intangible medium that is capable of storing, encoding or carrying instructions for execution by the machine, and includes digital or analog communications signals or other intangible media to facilitate communication of such software.

As used herein, “communicatively coupled between” means that the entities on either of the coupling must communicate through an item therebetween and that those entities cannot communicate with each other without communicating through the item.

### Additional Notes

The above detailed description includes references to the accompanying drawings, which form a part of the detailed description. The drawings show, by way of illustration but not by way of limitation, specific embodiments in which the disclosure can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a,” “an,” “the,” and “said” are used when introducing elements of aspects of the disclosure or in the embodiments thereof, as is common in patent documents, to include one or more than one or more of the elements, independent of any other instances or usages of “at least one” or “one or more.” In this document, the term “or” is used to refer to a nonexclusive or, such that “A or B” includes “A but not B,” “B but not A,” and “A and B,” unless otherwise indicated.

In the appended aspects, the terms “including” and “in which” are used as the plain-English equivalents of the respective terms “comprising” and “wherein.” Also, in the following aspects, the terms “comprising,” “including,” and “having” are intended to be open-ended to mean that there may be additional elements other than the listed elements, such that after such a term (e.g., comprising, including, having) in a aspect are still deemed to fall within the scope of that aspect. Moreover, in the following aspects, the terms “first,” “second,” and “third,” and so forth, are used merely as labels, and are not intended to impose numerical requirements on their objects.

Embodiments of the disclosure may be implemented with computer-executable instructions. The computer-executable instructions (e.g., software code) may be organized into one or more computer-executable components or modules. Aspects of the disclosure may be implemented with any number and organization of such components or modules. For example, aspects of the disclosure are not limited to the specific computer-executable instructions or the specific components or modules illustrated in the figures and described herein. Other embodiments of the disclosure may include different computer-executable instructions or components having more or less functionality than illustrated and described herein.

Method examples (e.g., operations and functions) described herein can be machine or computer-implemented at least in part (e.g., implemented as software code or instructions). Some examples can include a computer-readable medium or machine-readable medium encoded with instructions operable to configure an electronic device to perform methods as described in the above examples. An implementation of such methods can include software code, such as microcode, assembly language code, a higher-level language code, or the like (e.g., “source code”). Such software code can include computer readable instructions for performing various methods (e.g., “object” or “executable code”). The software code may form portions of computer program products. Software implementations of the embodiments described herein may be provided via an article of manufacture with the code or instructions stored thereon, or via a method of operating a communication interface to send data via a communication interface (e.g., wirelessly, over the internet, via satellite communications, and the like).

Further, the software code may be tangibly stored on one or more volatile or non-volatile computer-readable storage media during execution or at other times. These computer-readable storage media may include any mechanism that stores information in a form accessible by a machine (e.g., computing device, electronic system, and the like), such as, but are not limited to, floppy disks, hard disks, removable magnetic disks, any form of magnetic disk storage media, CD-ROMS, magnetic-optical disks, removable optical disks (e.g., compact disks and digital video disks), flash memory devices, magnetic cassettes, memory cards or sticks (e.g., secure digital cards), RAMs (e.g., CMOS RAM and the like), recordable/non-recordable media (e.g., read only memories (ROMs)), EPROMS, EEPROMS, or any type of media suitable for storing electronic instructions, and the like. Such computer readable storage medium coupled to a computer system bus to be accessible by the processor and other parts of the OIS.

In an embodiment, the computer-readable storage medium may have encoded a data structure for a treatment planning, wherein the treatment plan may be adaptive. The data structure for the computer-readable storage medium may be at least one of a Digital Imaging and Communications in Medicine (DICOM) format, an extended DICOM format, a XML format, and the like. DICOM is an international communications standard that defines the format used to transfer medical image-related data between various types of medical equipment. DICOM RT refers to the communication standards that are specific to radiation therapy.

In various embodiments of the disclosure, the method of creating a component or module can be implemented in software, hardware, or a combination thereof. The methods provided by various embodiments of the present disclosure, for example, can be implemented in software by using standard programming languages such as, for example, C, C++, Java, Python, and the like; and combinations thereof. As used herein, the terms “software” and “firmware” are interchangeable, and include any computer program stored in memory for execution by a computer.

A communication interface includes any mechanism that interfaces to any of a hardwired, wireless, optical, and the like, medium to communicate to another device, such as a memory bus interface, a processor bus interface, an Internet connection, a disk controller, and the like. The communication interface can be configured by providing configuration parameters and/or sending signals to prepare the communication interface to provide a data signal describing the software content. The communication interface can be accessed via one or more commands or signals sent to the communication interface.

The present disclosure also relates to a system for performing the operations herein. This system may be specially constructed for the required purposes, or it may comprise a general purpose computer selectively activated or reconfigured by a computer program stored in the computer. The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

In view of the above, it will be seen that the several objects of the disclosure are achieved and other advantageous results attained. Having described aspects of the disclosure in detail, it will be apparent that modifications and variations are possible without departing from the scope of aspects of the disclosure as defined in the appended aspects. As various changes could be made in the above constructions, products, and methods without departing from the scope of aspects of the disclosure, it is intended that all matter contained in the above description and shown in the accompanying drawings shall be interpreted as illustrative and not in a limiting sense.

The above description is intended to be illustrative, and not restrictive. For example, the above-described examples (or one or more aspects thereof) may be used in combination with each other. In addition, many modifications may be made to adapt a particular situation or material to the teachings of the disclosure without departing from its scope. While the dimensions, types of materials and coatings described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended aspects, along with the full scope of equivalents to which such aspects are entitled.

Also, in the above Detailed Description, various features may be grouped together to streamline the disclosure. This should not be interpreted as intending that an unexpected disclosed feature is essential to any aspect. Rather, inventive subject matter may lie in less than all features of a particular disclosed embodiment. Thus, the following aspects are hereby incorporated into the Detailed Description, with each aspect standing on its own as a separate embodiment. The scope of the disclosure should be determined with reference to the appended aspects, along with the full scope of equivalents to which such aspects are entitled. Further, the limitations of the following aspects are not written in means-plus-function format and are not intended to be interpreted based on 35 U.S.C. § 112, sixth paragraph, unless and until such aspect limitations expressly use the phrase “means for” followed by a statement of function void of further structure.

The Abstract is provided to comply with 37 C.F.R. § 1.72(b), to allow the reader to quickly ascertain the nature of the technical disclosure. It is submitted with the understanding that it will not be used to interpret or limit the scope or meaning of the aspects.

1. A system for verifying a primary radiation dose profile generated by a radiation machine for providing a radiotherapy to a subject, the system comprising:
a cloud-computing device or networked devices configured to provide cloud-based services including:
a file service to receive image information of the subject produced by the radiation machine and information about the radiation machine;
a dose engine service to determine a secondary radiation dose profile using the received image information and the radiation machine information; and
a dose evaluation service to verify the primary radiation dose profile using the secondary radiation dose profile; and
a user interface configured to enable a client access to one or more of the cloud-based services via a communication network, and to output the verification of the primary radiation dose profile to a user or a process.

2. The system of claim 1, wherein the file service is configured to receive a DICOM file of the subject, and to parse the received DICOM file to extract the image information and the radiation machine information from the received DICOM file.

3. The system of claim 2, wherein the file service is configured to extract treatment plan information from the parsed DICOM file, the treatment plan information including the primary radiation dose profile.

4. The system of any of claim 2, wherein the DICOM file is a compressed or encrypted DICOM file, and wherein the file service is configured to decompress or decrypt the DICOM file, and to parse the decompressed or decrypted DICOM file.

5. The system of claim 2, further comprising a cloud storage configured to store the DICOM file of the subject, or the image information and the radiation machine information extracted from the DICOM file.

6. The system of claim 1, wherein the radiation machine information received by the file service includes one or more radiation beam parameters or one or more gantry parameters.

7. The system of claim 1, wherein the dose engine service is configured to apply a secondary dose algorithm to one or more of the image information or the radiation machine information to determine the secondary radiation dose, the secondary dose algorithm different from a primary dose algorithm used by the radiation machine to calculate the primary radiation dose profile.

8. The system of claim 7, wherein the user interface is configured to receive a user input of the secondary dose algorithm, or a user selection of the secondary dose algorithm from a plurality of candidate dose algorithms.

9. The system of claim 1, wherein the dose evaluation service is configured to determine a consistency metric between the primary radiation dose profile and the secondary radiation dose profile, and to verify the primary radiation dose profile based on the determined consistency metric.

10. The system of claim 9, wherein the dose consistency metric includes a relative difference between the primary radiation dose profile and the secondary radiation dose profile, and the dose evaluation service is configured to verify the primary radiation dose profile in response to the dose consistency metric falling within a specified range.

11. The system of claim 1, wherein the cloud-computing device or networked devices include access points configured to support simultaneous access to the cloud-based services by two or more clients.

12. The system of claim 11, wherein the cloud-based services further include a tenant management service configured to queue service requests from the two or more clients.

13. The system of claim 1, wherein the cloud-computing device or networked devices are configured to generate a radiation treatment plan for the subject using a beam model based on the verification of the primary dose profile.

14. A method for verifying a primary radiation dose profile generated by a radiation machine for providing a radiotherapy to a subject, the method comprising, via a cloud-computing device or networked devices:
receiving a DICOM file of the subject;
parsing the received DICOM file and extracting image information and information about the radiation machine from the received DICOM file;
determining a secondary radiation dose profile using the image information and the machine information; and
verifying the primary radiation dose profile using the secondary radiation dose profile.

15. The method of claim 14, wherein determining the secondary radiation dose profile includes applying a secondary dose algorithm to one or more of the image information or the radiation machine information, the secondary dose algorithm different from a primary dose algorithm used by the radiation machine to calculate the primary radiation dose profile.

16. The method of claim 15, comprising receiving a user input of the secondary dose algorithm, or a user selection of the secondary dose algorithm from a plurality of candidate dose algorithms.

17. The method of claim 14, wherein verifying the primary radiation dose profile includes:
determining a consistency metric between the primary radiation dose profile and the secondary radiation dose profile; and
verifying the primary radiation dose profile in response to the determined consistency metric satisfying a condition.

18. The method of claim 17, comprising queuing service requests from the two or more clients simultaneously accessing to the cloud-based services.

19. The method of claim 14, comprising generating a radiation treatment plan for the subject using a beam model based on the verification of the primary dose profile.

20. A non-transitory machine-readable storage medium that includes instructions that, when executed by one or more processors of a machine, cause the machine to perform operations comprising:
receiving a DICOM file of a subject, the DICOM file generated by a radiation machine for providing a radiotherapy to the subject;
parsing the received DICOM file and extracting image information and information about the radiation machine from the received DICOM file;
determining a secondary radiation dose profile using the image information and the machine information; and
verifying the primary radiation dose profile using the secondary radiation dose profile.

21. The non-transitory machine-readable storage medium of claim 20, wherein the operation of determining the secondary radiation dose profile includes applying a secondary dose algorithm to one or more of the image information or the radiation machine information, the secondary dose algorithm different from a primary dose algorithm used by the radiation machine to calculate the primary radiation dose profile.

22. The non-transitory machine-readable storage medium of claim 21, wherein the operations comprise receiving a user input of the secondary dose algorithm, or a user selection of the secondary dose algorithm from a plurality of candidate dose algorithms.

23. The non-transitory machine-readable storage medium of claim 20, wherein the operation of verifying the primary radiation dose profile includes:
determining a consistency metric between the primary radiation dose profile and the secondary radiation dose profile; and
verifying the primary radiation dose profile in response to the determined consistency metric satisfying a condition.

24. The non-transitory machine-readable storage medium of claim 23, wherein the operations comprise queuing service requests from the two or more clients simultaneously accessing to the cloud-based services.

25. The non-transitory machine-readable storage medium of claim 23, wherein the operations comprise generating a radiation treatment plan using a beam model based on the verification of the primary dose profile.
